(19) United States
(12) Patent Application Publication
(10) Pub. No.: US 2024/0335469 A1
(43) Pub. Date: Oct. 10, 2024

(54) READY-TO-USE PHARMACEUTICAL COMPOSITIONS OF POTASSIUM PHOSPHATES INJECTION

(71) Applicant: AMNEAL EU, LIMITED, Tipperary County (IE)

(72) Inventors: Piyush Amrutlal Patel, Ahmedabad (IN); Chiragkumar Girishbhai Siddhapara, Ahmedabad (IN); Aditya Maheshchandra Patel, Ahmedabad (IN); Amarender Raddy Donthisi, Hyderabad (IN); Srinivas Ramachandra Rao Kone, Monroe Towmship, NJ (US)

(73) Assignee: AMNEAL EU, LIMITED, Tipperary County (IE)

(21) Appl. No.: 18/204,475

(22) Filed: Jun. 1, 2023

(30) Foreign Application Priority Data
Apr. 4, 2023 (IN) IN202321025397

(51) Int. Cl. A61K 33/42; A61K 9/00; A61K 47/02

(52) U.S. Cl. CPC A61K 33/42; A61K 9/0019; A61K 47/02

(57) ABSTRACT

The present invention relates to a ready-to-use injection compositions of potassium phosphates in sodium chloride and methods for preparing the same. The ready-to-use injection compositions contain 15 mmol/250 mL of phosphorus, 22 mEq/250 mL of potassium, equal or less than about 25 mcg/mL of aluminum and exhibit an osmolality value of from about 250 mOsmol/kg to about 450 mOsmol/kg. The ready-to-use injection compositions of the disclosure do not require any further reconstitution step prior to use.

### RELATED APPLICATIONS

This application claims priority to Indian Patent Application No. 202321025397, filed on Apr. 4, 2023.

### FIELD OF INVENTION

The present invention relates to stable ready-to-use injection compositions of potassium phosphates in sodium chloride and methods for preparing the same. The ready-to-use injection compositions provide 0.06 mmol/mL (15 mmol/250 mL) of phosphorus, 0.088 mEq/mL (22 mEq/250 mL) of potassium, less than or equal to about 25 mcg/mL of aluminum and exhibit an osmolality value of from about 250 mOsmol/kg to about 450 mOsmol/kg. The ready-to-use injection compositions of the disclosure do not require any further reconstitution step prior to use.

### BACKGROUND OF INVENTION

Potassium Phosphates Injection currently approved by the U.S. Food and Drug Administration (FDA) is available in three presentations as concentrated solutions comprising phosphorus 3 mmol/mL and potassium 4.4 mEq/mL in 5 & 15 mL and 50 ml vials. Potassium Phosphates, a replacement phosphorus product, is approved as a source of phosphorus in intravenous fluids to correct hypophosphatemia and for parenteral nutrition in adults and pediatric patients when oral or enteral replacement is not possible, insufficient, or contraindicated. Concentrated solution of the currently approved potassium phosphates product requires dilution with 0.9% sodium chloride solution or 5% dextrose solution before administration. The dilution requires specialized aseptic technique or work areas, e.g., laminar flow hood (or an equivalent clean air compounding area). Further, the diluted solution should be used immediately after preparation, however if storage is necessary, the solution is stable for a maximum of 4 hours at a temperature of from about 20° C. to 25° C. (68° F. to 77° F.) or 14 days under refrigeration storage temperature of from about 2° C. to about 8° C. (36° F. to 46° F.).

Additional disadvantages of the concentrated solution include variation in the pH of the diluted formulation with the choice of the diluent. Also, there is potential risk of contamination and dosing errors associated with the dilution.

A potential problem with the marketed potassium phosphates injection products is the aluminum content, especially after long-time storage. Parenteral nutrition additive solutions are generally contaminated with aluminum. Research indicates that patients with impaired kidney function, including neonates, who receive parenteral levels of aluminum at greater than 4 to 5 mcg/kg/day accumulate aluminum at levels associated with central nervous system and bone toxicity. To limit the risk of aluminum toxicity, the U.S. Food and Drug Administration (FDA) has rules limiting the total aluminum content in intravenous formulations. Since some preparations of phosphates solutions can contain up to 15,000 mcg/L of aluminum, the total daily aluminum limit may be exceeded. According to U.S. Food and Drug Administration (FDA) rules, aluminum content of large volume parenteral (LVP) drug products used in Total Parenteral Nutrition (TPN) therapy must not exceed 25 mcg/L.

Marketed potassium phosphates injection product is suitable for intravenous infusion into a central or peripheral vein after dilution. Osmolality of intravenous infusion is one of the key factors which can cause phlebitis, especially when given via peripheral vein. Most cited human study of osmolality-induced phlebitis shows that intravenous solution with osmolality of less than 450 mOsmol/kg exhibits lowest risk of phlebitis, followed by moderate risk of phlebitis with intravenous administration of a solution with an osmolality of 450 to 600 mOsmol/kg, and highest risk of phlebitis occurred with solutions with osmolality over 600 mOsmol/kg. This research was key in establishing 500 mOsmol/kg as the outer limit of peripheral vein tolerance. Also, the Infusion Nursing Society has recommended upper osmolality limit under 500 mOsmol/kg for large volume injections administration through peripheral vein for prevention of vascular damage. Hypertonic injection solutions with an osmolality >600 mOsmol/kg have been reported to possibly cause crenation (shrivelling up) of red blood cells and significant pain.

There is a need for a stable ready-to-use potassium phosphates injection composition providing 1) improved patient compliance, 2) reduced dosage errors, 3) storage stability in diluted form, and 4) reduced risk of contamination and compounding. There is a need for ready-to-use potassium phosphates injection compositions comprising aluminum content of less than or equal to about 25 mcg/mL, and a desired osmolality value of from about 250 mOsmol/kg to about 450 mOsmol/kg to reduce the risk for phlebitis. The present disclosure provides stable ready-to-use potassium phosphates injection compositions that minimize human error and microbial infections associated with dilution, while stabilizing potassium phosphates during storage under room temperature conditions.

### SUMMARY OF THE INVENTION

The present invention relates to a ready-to-use potassium phosphates injection composition comprising monobasic potassium phosphate, dibasic potassium phosphate, and an tonicity agent. The composition, on storage for at least about 6 months at 25° C.±2° C./40% RH±5% RH, comprises an assay of each of the monobasic potassium phosphate and the dibasic potassium phosphate of from about 90% w/w to about 110% w/w compared to their corresponding initial assays, and maintains an osmolality value of from about 250 mOsmol/kg to about 450 mOsmol/kg.

In certain embodiments, the composition comprises 0.088 mEq/mL of potassium and 0.06 mmol/mL of phosphorus.

In certain embodiments, the composition comprises a fill volume of about 250 mL.

In certain embodiments, the tonicity agent is selected from the group consisting of sodium chloride, potassium chloride, dextrose, sugars, sugar alcohols, glucose, xylitol, fructose, glycerol, sorbitol, mannitol, mannose, calcium chloride, magnesium chloride, other inorganic salts, and mixtures thereof.

In certain embodiments, the composition is an aqueous composition for intravenous administration.

In certain embodiments, the composition comprises a fill volume of about 250 mL.

In certain embodiments, the composition maintains a pH of from about 5.5 to about 7.5.

In certain embodiments, the composition maintains an osmolality value of from about 400 mOsmol/kg to about 410 mOsmol/kg.

In certain embodiments, the composition exhibits an osmolarity of about 0.455 mOsmol/mL.

In certain embodiments, the composition is supplied in a bag that is further packed in an aluminum overwrap, overpouch or equivalent thereof.

In certain embodiments, the composition is terminally sterilized.

In certain embodiments, the composition further comprises less than or equal to about 15 mcg/L of aluminum.

In certain embodiments, the composition after storage for at least about 12 months at 25° C.±2° C./40% RH±5% RH, comprises an assay of each of the monobasic potassium phosphate and the dibasic potassium phosphate of from about 90% w/w to about 110% w/w compared to their corresponding initial assays, and maintains an osmolality value of from about 250 mOsmol/kg to about 450 mOsmol/kg.

In certain embodiments, the disclosure provides a ready-to-use potassium phosphates injection composition comprising monobasic potassium phosphate, dibasic potassium phosphate, and an tonicity agent. The composition, on storage for up to about 6 months at 40° C.±2° C./NMT 25% RH, comprises an assay of each of the monobasic potassium phosphate and the dibasic potassium phosphate of from about 90% w/w to about 110% w/w, compared to their corresponding initial assays, and maintains an osmolality value of from about 250 mOsmol/kg to about 450 mOsmol/kg.

In certain embodiments, the disclosure provides a ready-to-use potassium phosphates injection composition comprising monobasic potassium phosphate, dibasic potassium phosphate, and an tonicity agent. The composition, on storage for at least about 6 months at 5° C.±3° C., comprises an assay of each of the monobasic potassium phosphate and the dibasic potassium phosphate of from about 90% w/w to about 110% w/w, compared to their corresponding initial assays, and maintains an osmolality value of from about 250 mOsmol/kg to about 450 mOsmol/kg.

### DETAILED DESCRIPTION OF INVENTION

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing,” or “comprising” are interchangeable, and one of skill in the art is cognizant that these terms are open-ended terms.

As used herein, the terms “comprise,” “comprises”, and “comprising” are to be interpreted inclusively rather than exclusively. The words “consist,” “consisting,” and its variants, are to be interpreted exclusively, rather than inclusively.

As used herein, the term “and/or” refers to and encompasses any and all possible combinations of one or more of associated listed items.

As used herein, and unless otherwise specified, the terms “about” or “approximately.” mean an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

The terms “Potassium Phosphates” and “a mixture/combination of monobasic and dibasic potassium phosphates” as used interchangeably herein, refer to a mixture of salts of potassium and phosphate ions.

As used herein, unless otherwise specified, the term “stable” refers to stable injectable compositions of potassium phosphates that do not exhibit more than 5% change in assay from their initial assay. Stable ready-to-use injectable compositions of the disclosure retain their properties without loss of potency of the active and maintain their physical characteristics over time with minimal degradation. In some respects, “stable composition” refers to pharmaceutical compositions that comply with ICH guidelines as generally followed in the pharmaceutical arts. In some respects, the “stable composition” contains from about 90% w/w to about 110% w/w of assay, based on the initial assay or labeled amount.

The term “treating” or “treatment” is meant to encompass administering to a subject a compound of the present disclosure for the purposes of prophylaxis and/or amelioration of one or more symptoms of a disease or disorder, including palliative care. As used herein, an “effective amount” or a “therapeutically effective amount” is used interchangeably and refers to an amount of a pharmaceutical composition of the present disclosure which provides the desired treatment of a subject. As would be appreciated by one of ordinary skill in the art, the therapeutically effective amount of the present pharmaceutical compositions to treat a given disease, disorder or condition will vary from subject to subject, depending on factors such as age, general condition of the subject, the severity of the condition being treated, the particular compound and/or formulation administered, and the like. An appropriate therapeutically effective amount of the present pharmaceutical compositions suitable for any individual subject can be readily determined by one of ordinary skill in the art from the information provided herein.

As used herein the term “long-term storage conditions” refers to at least about 6 month storage (e.g., up to about 12 months or higher) of the ready-to-use potassium phosphates injection composition at 25° C.±2° C. and 40%±5% relative humidity (RH) or 30° C.±2° C. and 35%±5% relative humidity (RH).

As used herein the term “intermediate storage conditions” refers to at least about 6 month storage (e.g., up to about 12 months or higher) of the ready-to-use potassium phosphates injection composition at 30° C.±2° C. and 65%±5% RH. There is no intermediate storage condition if the long term storage condition is 30° C.±2° C. and 35%±5% relative humidity (RH).

As used herein the term “accelerated storage conditions” refers to up to about 6 month storage of the ready-to-use potassium phosphates injection composition at 40° C.±2° C. and NMT 25% RH.

The terms “room temperature” and “controlled room temperature,” as used interchangeably herein, refer to 20° C. to about 25° C.

As used herein the term “freezing storage conditions” refers to at least about 6 month (e.g., up to about 9 months, up to about 12 months) storage of the ready-to-use potassium phosphates injection composition at −20° C.±5° C.

As used herein the term “refrigeration storage conditions” refers to at least 6 month (e.g., up to 9 months, up to or 12 months, or up to 5 years) storage of the ready-to-use potassium phosphates injection composition at 5° C.±3° C.

The terms “osmolality” and “measured osmolality,” as used interchangeably herein, refer to the number of solute particles in 1 kg of water solution, e.g., mOsmol/kg solution, e.g., concentration in moles/kg water solution. Osmolality is generally measured using an osmometer—either a freezing point depression osmometer or a vapour pressure depression osmometer.

The terms “osmolarity” and “calculated osmolarity,” as used interchangeably herein, refer to the number of solute particles in 1L of water solution, e.g., concentration in moles/L of water solution.

As used herein, unless otherwise specified, the term “osmolar gap” refers to difference between calculated osmolarity and measured osmolarity.

As used herein, unless otherwise specified, the term “F0” refers to time in minutes for a specified temperature that provides the same thermal lethality at 121° C. in one minute. F0is calculated using the following general formula: F0=ΔT=10 (T−121/10); wherein T is the temperature of sterilization, ΔT=1 minute, and Z=10° C.

As used herein, unless otherwise specified, the term “thermal lethality” refers to time in minutes necessary for the mitigation of inadvertent contamination during aseptic processing at a specified temperature.

As used herein, and unless otherwise specified, the term “ready-to-use” refers to a sterile drug solution that does not require any further dilution or reconstitution prior to administration/infusion to a patient in need thereof. The term “ready-to-use” is synonymous with “premixed,” “ready-to-infuse,” and “ready-to-administer” stable pharmaceutical composition of present invention.

As used herein the term “aqueous” should be accorded its normal meaning which may mean the composition, formulation or material that contains water as a solvent or medium.

The terms “flexible plastic container” and “flexible plastic intravenous container,” as used interchangeably herein, refer to a flexible container in which injection compositions for intravenous administration can be packaged for use. Flexibility of the intravenous container provides maximum drainage and minimum residual volume of the infusion liquid to minimize dosing error. Additionally, the flexible intravenous container does not require external venting to empty the container. As used herein, and unless otherwise specified, the flexible plastic container may include but is not limited to a flexible bag or a flexible bottle.

As used herein, and unless otherwise specified, the term “terminal sterilization” is defined as the process whereby product is sterilized within its sterile barrier system. The terminal sterilization process is considered a manufacturing process step itself and usually takes place at, or near, the end of the manufacturing process. An autoclave is commonly used to accomplish terminal heat sterilization of drug products in their final packaging. Typical autoclave cycles in the pharmaceutical industry to achieve terminal sterilization of the final product include heating to about 121° C. for a predefined time and cycles (e.g., 30-90 minutes and 4 cycles or less).

The terms “isotonic agent” and “tonicity agent,” as used interchangeably herein, refer to chemical compounds in a pharmaceutical composition that modify osmotic pressure of the pharmaceutical composition so that the osmotic pressure becomes closer to that of human plasma.

As used herein, and unless otherwise specified, the term “pH” refers to a measure of effective concentration of hydrogen ions in a solution (e.g., approximately related to the molarity of by H+ by pH=−log [H+]).

As used herein, and unless otherwise specified, the term “shelf-life” refers to a time period during which a pharmaceutical composition may be stored and remains suitable for use.

As used herein, and unless otherwise specified, the term “phlebitis” refers to inflammation of the walls of a vein.

As used herein, and unless specified, the term “hypophosphatemia” refers to a serum phosphate concentration of <2.5 mg/dL (0.81 mmol/L).

As used herein, and unless specified, the term “hyperphosphatemia” refers to a serum phosphate concentration of between 2.5 mg/dl to 4.5 mg/dL.

As used herein, and unless specified, the term “hyperkalemia” refers to a serum potassium concentration of greater than 5 mEq/L to 5.5 mEq/L.

As used herein, and unless specified, the term “hypokalemia” refers to a serum potassium concentration of less than 3.5 mEq/L (3.5 mmol/L).

As used herein, and unless specified, the term “hypercalcemia” refers to a total serum calcium concentration of greater than 10.5 mg/dL (>2.60 mmol/L) or ionized serum calcium if greater than 5.2 mmol/dL (1.30 mmol/L).

As used herein, and unless specified, the term “hypocalcemia” refers to a total serum calcium concentration of less than 8.8 mg/dL (2.2 mmol/L).

### Potassium Phosphates in Sodium Chloride Injection Compositions

The present invention relates to ready-to-use (e.g., ready-to-infuse) injection compositions of potassium phosphates in sodium chloride. The ready-to-use//ready-to-infuse injection compositions contain 15 mmol/250 mL (0.06 mmol/mL) of phosphorus, 22 mEq/250 mL (0.088 mEq/mL) of potassium, less than or equal to about 25 mcg/ml of aluminum and exhibit an osmolality value of from about 250 mOsmol/kg to about 450 mOsmol/kg. The ready-to-use injection compositions containing 15 mmol/250 mL (0.06 mmol/mL) of phosphorus, 22 mEq/250 mL (0.088 mEq/mL) of potassium, and less than or equal to about 25 mcg/mL of aluminum exhibit an osmolarity value of from about 250 mOsmol/L to about 500 mOsmol/L. The ready-to-use injection compositions of the disclosure do not require any further reconstitution/dilution step prior to use.

In one embodiment, the stable ready-to-use/ready-to-infuse injection compositions of the disclosure contain 4.48 mg/mL of monobasic potassium phosphate and 4.72 mg/ml of dibasic potassium phosphate in 0.9% sodium chloride solution (potassium phosphates in sodium chloride injection). The stable ready-to-use injection compositions of the disclosure exhibit a pH of from about 5.8 to about 7.2 (e.g., about 6.6), and osmolarity of about 0.455 mOsmol/mL (455 mOsmol/L).

In certain embodiments, the ready-to-use/ready-to-infuse injection compositions of the disclosure exhibit storage stability for at least about 6 months. In certain embodiments, the ready-to-use injection compositions of the disclosure exhibit storage stability from about 6 months to about 5 years. In certain embodiments, the ready-to-use injection compositions of the disclosure (e.g., potassium phosphates injection composition in flexible plastic bag with aluminum overwrap or over pouch) on storage under long-term storage conditions at 25° C.±2° C. and not more than (NMT) 40%±5% relative humidity (RH), exhibit storage stability of from about 6 months to about 5 years.

In certain embodiments, the ready-to-use//ready-to-infuse injection compositions of the disclosure (e.g., potassium phosphates injection composition in flexible plastic bag with aluminum overwrap or over pouch) on storage under accelerated storage conditions at 40° C.±2° C. and 25% RH, exhibit storage stability of up to about 6 months.

In certain embodiments, the ready-to-use/ready-to-infuse injection compositions of the disclosure (e.g., potassium phosphates injection composition in flexible plastic bag with aluminum overwrap or over pouch) on storage under refrigerated storage conditions at 5° C.±3° C., exhibit storage stability of at least about 6 months or more, e.g., up to about 5 years.

In certain embodiments, the ready-to-use/ready-to-infuse injection compositions of the disclosure (e.g., potassium phosphates injection composition in flexible plastic bag with aluminum overwrap or over pouch) on storage under freezing storage conditions at −20° C.±5° C., exhibit storage stability of at least about 6 months, e.g., up to about 9 months, up to about 12 months.

In certain embodiments, the ready-to-use/ready-to-infuse injection compositions of the disclosure (e.g., potassium phosphates injection composition in flexible plastic bag with aluminum overwrap or over pouch) exhibit long-term storage stability for about 6 months, about 9 months, about 1 year, about 15 months, about 18 months, about 21 months, about 2 years, about 3 years, about 4 years, about 5 years or any intermediate periods therein.

In certain embodiments, the ready-to-use/ready-to-infuse injection compositions of the disclosure comprises potassium phosphates in sodium chloride injection stored in flexible plastic container.

In certain embodiments, the potassium phosphates in sodium chloride injection compositions of the disclosure exhibit osmolality value of from about 250 mOsmol/kg to about 450 mOsmol/kg and an osmolarity value of from about 250 mOsmol/L to about 500 mOsmol/L.

In certain embodiments, the ready-to-use/ready-to-infuse injection compositions of the disclosure exhibit an osmolar gap between measured osmolarity (osmolality) and calculated osmolarity. In certain embodiments, the osmolar gap is from 0.1 to about 100. In certain embodiments, the osmolar gap is from 0.1 to about 50. In certain embodiments, the injection compositions of the disclosure exhibit no osmolar gap (e.g., exhibit same measure and calculated osmolarity).

According to the USP <785>, the osmolality of blood ranges from 285 and 310 mOsmol/kg with average physiological osmolality of about 297.5 mOsmol/kg. Infusion Nursing Society has recommended upper osmolality limit under 500 mOsmol/kg for large volume injections administration through peripheral vein for prevention of vascular damage. The ready-to-infuse large volume injection compositions of present disclosure minimize risk of phlebitis when administered through peripheral vein. The osmolality value of from about 250 mOsmol/kg to about 450 mOsmol/kg, and osmolarity value of from about 250 mOsmol/L to about 500 mOsmol/L is maintained after storage of the ready-to-use injection compositions of the disclosure for at least about 6 months under accelerated storage conditions of 40° C.±2° C./NMT 25% RH, long-term storage conditions at 25° C.±2° C./40% RH±5% RH, refrigeration condition of 5° C.±3° C., and freezing condition of −20° C.±5° C.

In certain embodiments, the ready-to-use/ready-to-infuse potassium phosphates in sodium chloride injection compositions exhibit an osmolality of from about 250 mOsmol/kg to about 450 mOsmol/kg. from about 280 mOsmol/kg to about 440 mOsmol/kg, from about 310 mOsmol/kg to about 430 mOsmol/kg, from about 330 mOsmol/kg to about 420 mOsmol/kg, and from about 370 mOsmol/kg to about 410 mOsmol/kg. In certain embodiments, the ready-to-use potassium phosphates in sodium chloride injection compositions exhibit an osmolality of about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450 mOsmol/kg, or any intermediate values therein. In certain embodiments, the ready-to-use potassium phosphates in sodium chloride injection compositions of the disclosure exhibit an osmolality of about 405 mOsmol/kg.

The osmolality of a solution is commonly determined by measuring the depression in freezing point of the injection solution using an osmometer. The osmometer comprises a means of cooling the container used for the measurement: a resistor sensitive to temperature (thermistor), with an appropriate current or potential difference measurement device that may be graduated in temperature change or in osmolality: and a means of mixing the sample. The sample solution is prepared by taking a sample bag, shaking the bag to shake the contents well and withdrawing about 5 mL of sample into suitable clean and dried glass container as per sample withdrawal procedure. About 0.25 mL of sample solution is transferred into osmometer cuvette and osmolality is measured and recorded in mOsmol/kg.

In certain embodiments, the ready-to-use/ready-to-infuse potassium phosphates in sodium chloride injection compositions exhibit an osmolarity of from about 250 mOsmol/L to about 500 mOsmol/L, from about 280 mOsmol/L to about 440 mOsmol/L, from about 310 mOsmol/L to about 430 mOsmol/L, from about 330 mOsmol/L to about 420 mOsmol/L, and from about 370 mOsmol/L to about 410 mOsmol/L. In certain embodiments, the ready-to-use potassium phosphates in sodium chloride injection compositions exhibit an osmolarity of about 250), about 260), about 270), about 280), about 290, about 300, about 310, about 320, about 330, about 340, about 350, about 360, about 370, about 380, about 390, about 400, about 405, about 410, about 415, about 420, about 425, about 430), about 435, about 440), about 445, about 450), about 455, about 460), about 465, about 470), about 475, about 480), about 485, about 490, about 495, about 500 mOsmol/L or any intermediate values therein. In certain embodiments, the ready-to-use potassium phosphates in sodium chloride injection compositions of the disclosure exhibit an osmolarity of about 455 mOsmol/L (0.455 mOsmol/mL).

In certain embodiments, the ready-to-use/ready-to-infuse injection compositions of the disclosure exhibit an osmolar gap between measured osmolarity and calculated osmolarity. In certain embodiments, the osmolar gap is from 0.1 to about 100. In certain embodiments, the osmolar gap is from 0.1 to about 50. In certain embodiments, the osmolar gap is about 10, about 15, about 20, about 25, about 30, about 35, about 40, about 45, about 50, about 55, about 60), about 65, about 70, about 75, about 80, about 85, about 90, about 95, about 100, or any intermediate values therein. In certain embodiments, the injection compositions of the disclosure exhibit no osmolar gap (e.g., exhibit same measured and calculated osmolarity).

In certain embodiments, the ready-to-use/ready-to-infuse potassium phosphates in sodium chloride injection compositions of the disclosure comprise 4.48 mg/mL of monobasic potassium phosphate and 4.72 mg/mL of dibasic potassium phosphate, and less than or equal to about 25 mcg/L of aluminum.

In certain embodiments, the ready-to-use/ready-to-infuse potassium phosphates in sodium chloride injection compositions of the disclosure comprise aluminum in an amount of less than or equal to about 15 mcg/L. In certain embodiments, the aluminum content in the ready-to-use potassium phosphates in sodium chloride injection compositions of the disclosure is from 0.1 to about 25 mcg/L. In certain embodiments, the ready-to-use potassium phosphates in sodium chloride injection compositions of the disclosure are free of aluminum. In certain embodiments, the aluminum content in ready-to-use potassium phosphates in sodium chloride injection compositions of the disclosure is less than or equal to about 1 mcg/L, about 2 mcg/L, about 3 mcg/L, about 4 mcg/L, about 5 mcg/L, about 6 mcg/L, about 7 mcg/L, about 8 mcg/L, about 9 mcg/L, about 10 mcg/L mc/L, about 11 mcg/L, about 12 mcg/L, about 13 mcg/L, about 14 mcg/L, about 15 mcg/L, about 16 mcg/L, about 17 mcg/L, about 18 mcg/L, about 19 mcg/L, about 20 mcg/L, about 21 mcg/L, about 22 mcg/L, about 23 mcg/L, about 24 mcg/L, about 25 mcg/L, or any intermediate values therein. In certain embodiments, the aluminium content in the potassium phosphates in sodium chloride injection compositions of the disclosure is less than or equal to about 25 mcg/L after storage under accelerated storage conditions of 40° C.±2° C./NMT 25% RH, long-term storage conditions at 25° C.±2° C./40% RH±5% RH, refrigeration condition of 2° C. to 8° C. (e.g., 5° C.±3° C.) and/or freezing condition of −20° C.±5° C. for at least about 6 months.

In certain embodiments, the ready-to-use/ready-to-infuse potassium phosphates in sodium chloride injection compositions of the disclosure are stable at intermediate storage conditions (30° C.±2° C. and 65±5% RH) for at least about 6 months (e.g., 12 months or more).

In certain embodiments, the ready-to-use/ready-to-infuse potassium phosphates in sodium chloride injection compositions are supplied in the fill volume of from about 100 mL to about 500 mL. In certain embodiments, the fill volume is about 100 ml, about 150 ml, about 200 mL, about 250 mL, about 300 mL, about 350 mL, about 400 mL, about 450 mL. about 500 mL, or any intermediate volumes therein. In certain embodiments, the fill volume of the ready-to-use potassium phosphates in sodium chloride injection compositions of the disclosure is about 250 mL.

In certain embodiments, the ready-to-use//ready-to-infuse potassium phosphates in sodium chloride injection is an intravenous injection composition supplied in flexible plastic intravenous containers as opposed to rigid or semi-rigid intravenous containers.

In certain embodiments, the flexible plastic intravenous container is a closed system fully collapsible intravenous bag or a bottle. Flexibility of the intravenous container provides maximum drainage and minimum residual volume of the infusion liquid to minimize dosing error. Additionally, the flexible intravenous container does not require external venting to empty the container.

In certain embodiments, the flexible plastic intravenous container is a closed system fully collapsible intravenous bag.

In certain embodiments, the flexible plastic container is further packed in aluminum overwrap, overpouch or equivalent thereof.

Examples of commercially available intravenous bags include, but are not limited to EXCEL®, VISIV®, NEXCEL®, INTERVIA®, TECHNOFLEX®, GALAXY®, SOLOMIX®, STEDIM71®, STEDIM®100, VIAFLEX®, VIAFLO®, ADDEASE®, ADD-VANTAGE®, DUPLEX®, FIRST CHOICE®, PROPYFLEX®, FREEFLEX®, POLYCINE®, MAGIFLEX® and BFS®.

In certain embodiments, the flexible plastic intravenous container is a non-plasticized film containing polypropylene and thermoplastic elastomers (FREEFLEX®).

In certain embodiments, the flexible plastic intravenous container is a multilayer polyolefin/styrene block copolymer based film (POLYCINE®).

In certain embodiments, the flexible plastic intravenous container is a multilayer film bag comprising polypropylene polymer or polypropylene based polyolefin polymer: and styrene-ethylene-butylene block copolymer (TECHNOFLEX®).

In certain embodiments, the flexible plastic intravenous container is a multilayer sheeting composed of polypropylene (PP), polyamide (PA) and polyethylene (PE) (VIAFLO®).

In certain embodiments, the flexible plastic intravenous container is non-DEHP (Diethylhexyl-phthalate), non-PVC (polyvinyl chloride) and latex-free.

In certain embodiments, the ready-to-use potassium/ready-to-infuse phosphates injection compositions exhibit a pH range of from about 5.5 to about 7.5. In certain embodiments, the ready-to-use potassium phosphates injection compositions exhibit a pH range of from about 5.8 to about 7.2. In certain embodiments, the ready-to-use potassium phosphates injection compositions exhibit a pH range of about 5.6 to about 7.4, about 5.7 to about 7.3, about 5.8 to about 7.2, about 5.9 to about 7.1, about 6.0 to about 7.0, or any intermediate values therein. In certain embodiments, the ready-to-use potassium phosphates injection compositions exhibit a pH of about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, or any intermediate values therein.

In certain embodiments, the ready-to-use/ready-to-infuse potassium phosphates in sodium chloride injection compositions comprises 4.48 mg/mL of monobasic potassium phosphate and 4.72 mg/mL of dibasic potassium phosphate, wherein the composition exhibits a pH of from about 5.5 to about 7.5. In certain embodiments, the composition exhibits a pH of from about 5.8 to about 7.2.

In certain embodiments, the potassium phosphates injection compositions of the disclosure comprise at least one tonicity/isotonic agent selected from the group comprising sodium chloride, potassium chloride, dextrose, sugars, sugar alcohols, glucose, xylitol, fructose, glycerol, sorbitol, mannitol, mannose, calcium chloride, magnesium chloride, other inorganic salts, and mixtures thereof. In certain embodiments, the tonicity agent is sodium chloride. In certain embodiments, the tonicity agent is present from about 5 mg/mL to about 20 mg/mL: from about 5 mg/mL to about 10 mg/mL, about 6 mg/mL, about 7 mg/mL, about 8 mg/mL, about 9 mg/mL, or any intermediate values therein. In certain embodiments, the tonicity agent is sodium chloride in an amount of about 9 mg/mL (0.9% sodium chloride).

In certain embodiments, the ready-to-use/ready-to-infuse potassium phosphates injection compositions of the disclosure are terminally sterilized using typical autoclave cycles used in the pharmaceutical industry to achieve terminal sterilization of the final product. In certain embodiments, the ready-to-use potassium phosphates injection compositions of the disclosure are autoclaved at about 121° C. for about 15 minutes, about 30 minutes, about 60 minutes or more based on F₀ based cycle to assure the sterility assurance of the finished product. In certain embodiments, the potassium phosphates injection compositions of the present invention are autoclaved at about 121° C. using steam-air mixture for at least about 15 minutes (e.g., about 20 minutes).

### Method of Treatment

In certain embodiments, the disclosure provides methods of using ready-to-use/ready-to-infuse potassium phosphates (mixture/combination of monobasic and dibasic potassium phosphates) injection compositions as a phosphorus replacement product indicated as a source of phosphorus. The ready-to-use potassium phosphates injection compositions of the disclosure are administered in a treatment method comprising intravenous infusion to correct hypophosphatemia in adults and paediatric patients when oral or enteral replacement is not possible, insufficient, or contraindicated.

### Method of Administration to Correct Hypophosphatemia

The ready-to-use intravenous injection compositions of the disclosure comprise potassium phosphates in 0.9% sodium chloride injection. The serum potassium and calcium concentrations of the patient are checked prior to administration of the injection, and the calcium level is normalized before the administration of the ready-to-use potassium phosphates injection composition of the disclosure.

Potassium phosphates in 0.9% sodium chloride injection compositions of the disclosure are suitable only for administration to a patient with a serum potassium concentration less than 4 mEq/dL. For patients with potassium concentration of 4 mEq/dL or more, an alternative source of phosphorus is used.

The ready-to-use potassium phosphates in 0.9% sodium chloride injection is never infused with calcium-containing intravenous fluids.

In certain embodiments, the rate of administration is dependent on the patient and the specific institution policy. The dosage is dependent upon the individual needs of the patient, and the contribution of potassium and phosphorus from other sources.

The ready-to-use injection solution is visually inspected for particulate matter and discoloration prior to administration. Colored or cloudy solutions or solutions containing particulate matter are discarded.

### Dosage for Administration to Correct Hypophosphatemia

The ready-to-use potassium phosphates injection is only for administration to a patient with a serum potassium concentration less than 4 mEq/dL. The dosage is dependent upon individual needs of the patient, and the contribution of phosphorus and potassium from other sources.

### Initial or Single-Dose

Table 1 provides recommended initial or single dose of the ready-to-use potassium phosphates in 0.9% sodium chloride injection in intravenous fluids to correct hyperphosphatemia in adults and pediatric patients.

TABLE 1

| Serum Phosphorus | Phosphorus | Corresponding |
|---|---|---|
| Concentration^{a} | Dosage^{b,c} | Potassium Content |
| 1.8 mg/dL to lower end of the | 0.16 mmol/kg to | potassium 0.23 |
| reference range^{a} | 0.31 mmol/kg | mEq/kg to 0.46 |
| | | mEq/kg |
| 1 mg/dL to 1.7 mg/dL | 0.32 mmol/kg to | potassium 0.47 |
| | 0.43 mmol/kg | mEq/kg to 0.63 |
| | | mEq/kg |
| Less than 1 mg/dL | 0.44 mmol/kg to | potassium 0.64 |
| | 0.64 mmol/kg^{c} | mEq/kg to 0.94 |
| | | mEq/kg |

^{a}Serum phosphorus reported using 2.5 mg/dL as the lower end of the reference range for healthy adults and pediatric patients 12 months of age and older. Serum phosphorus reported using 4 mg/dL as the lower end of the reference range for preterm and term infants less than 12 months of age. Serum phosphorus concentrations may vary depending on the assay used and the laboratory reference range.
^{b}Weight is in terms of actual body weight. Limited information is available regarding dosing of patients significantly above ideal body weight; consider using an adjusted body weight for these patients.
^{c}up to a maximum of phosphorus 45 mmol (potassium 66 mEq) as a single dose.

### Intravenous Infusion Rate

Intravenous infusion rate of the ready-to-use potassium phosphates compositions of the disclosure depend upon route of administration, e.g., through a peripheral or central venous catheter route. Table 2 provides recommended infusion rates, peripheral or central venous catheter route, for adults and pediatric patients 12 years of age and older.

TABLE 2

| Intravenous Administration Route | Maximum Infusion Rate |
|---|---|
| Peripheral Venous Catheter | Phosphorus 6.8 mmol/hour |
| | (Potassium 10 mEq/hour) |
| Central Venous Catheter | Phosphorus 15 mmol/hour |
| | (Potassium 22 mEq/hour |

Continuous electrocardiogramhic (ECG) monitoring and infusion through central nervous catheter is recommended for infusion rates higher than the following:
Potassium 10 mEq/hour for adults and pediatric patients weighing 20 kg or more.
Potassium 0.5 mEq/kg/hour for adults and pediatric patients weighing less than 20 kg.

### EXAMPLES

The following examples illustrate the disclosure in a nonlimiting manner. Unless indicated to the contrary, the numerical parameters set forth herein can vary depending upon the desired properties sought to be obtained by the present disclosure.

### Example 1—Compositions of Potassium Phosphates in Sodium Chloride Injection

TABLE 3

| | Formulation # | | | | | |
|---|---|---|---|---|---|---|
| | 1 | 2 | 3 | 4 | 5 | 6 |
| Ingredients | mg./mL | mg./mL | mg./mL | mg./mL | mg./mL | mg./mL |
| Monobasic | 4.48 | 4.48 | 4.48 | 4.48 | 4.48 | 4.48 |
| potassium | | | | | | |
| phosphate | | | | | | |
| USP-NF | | | | | | |
| Dibasic | 4.72 | 4.72 | 4.72 | 4.72 | 4.72 | 4.72 |
| potassium | | | | | | |
| phosphate USP | | | | | | |
| Sodium | 9 | 8.5 | 7.5 | 6.5 | 5.5 | 4.5 |
| chloride | (0.9%) | (0.85%) | (0.75%) | (0.65%) | (0.55%) | (0.45%) |
| USP(% NaCl) | | | | | | |
| Water for | q.s. to | q.s. to | q.s. to | q.s. to | q.s. to | q.s. to |
| Injection USP | 1 mL | 1 mL | 1 mL | 1 mL | 1 mL | 1 mL |

### Process for Preparation

1. About 95% of the total volume of water for injection was taken in a manufacturing vessel and maintained at a temperature of about 15° C. to about 25° C.
2. Weighed quantity of sodium chloride was added to the water for injection USP of step 1 with continuous stirring until a clear solution was obtained.
3. Weighed quantity of dibasic potassium phosphate USP was added to the solution of step 2 with continuous stirring until a clear solution was obtained.
4. Weighed quantity of monobasic potassium phosphate USP-NF was added to the solution of step 3 with continuous stirring until clear solution was obtained.
5. Water for injection USP was added to the clear solution of step 4 to make up the volume to 100%.
6. The clear solution of step 5 was filtered using a 0.2μ filter to obtain a clear, colorless solution.
7. The clear, colorless solution of step 6 was filled in a flexible plastic bag and sealed.
8. The filled and sealed bag of step 7 was terminally sterilized and packed.

Table 4 provides initial data, e.g., osmolality, pH, and aluminium content, of Formulations 1-6.

TABLE 4

| | Formulation # | | | | | |
|---|---|---|---|---|---|---|
| Initial data | 1 | 2 | 3 | 4 | 5 | 6 |
| Osmolality | 401 | 380 | 363 | 335 | 303 | 270 |
| mOsmol/kg | | | | | | |
| Aluminum | 10 | 12 | 10 | 9 | 14 | 12 |
| Content | | | | | | |
| (mcg/mL) | | | | | | |
| pH | 6.651 | 6.624 | 6.636 | 6.60 | 6.64 | 6.65 |

Data from Table 4 illustrates that the ready-to-infuse injection composition 1-6 exhibit initial osmolality values of from about 250 mOsmol/kg to about 450 mOsmol/kg (e.g., from 270-401 mOsmol/kg) and aluminium content of less than or equal to about 25 mcg/mL (e.g., 14 mcg or less). The osmolality and aluminium content are expected to be maintained throughout the shelf life of at least about 6 months.

### Example 2—Stability of the Ready-To-Use Potassium Phosphates in 0.9% Sodium Chloride Injection Composition

Stability of three batches (Batch 1-3) of terminally sterilized Formulation 1 was determined under long-term, accelerated, refrigeration and freezing storage conditions.

Table 5 provides 6-month storage stability of Batch 1-3 of terminally sterilized Formulation 1 under accelerated storage conditions (40° C.±2° C./NMT 25% RH).

TABLE 5
Potassium phosphates in 0.9% sodium chloride injection, Phosphorus
15 mmol/250 mL and Potassium 22 mEq/250 mL (Formulation 1)

| | Batch 1 | | | Batch 2 | | | Batch 3 | | |
|---|---|---|---|---|---|---|---|---|---|
| Data. | Init | 3 M | 6 M | Init | 3 M | 6 M | Init | 3 M | 6 M |
| pH | 6.6 | 6.6 | 6.6 | 6.6 | 6.6 | 6.6 | 6.6 | 6.6 | 6.6 |
| Assay of | 101 | 101 | 102 | 101 | 101 | 101 | 102 | 101 | 102 |
| monobasic | | | | | | | | | |
| potassium | | | | | | | | | |
| phosphate | | | | | | | | | |
| (% w/w) | | | | | | | | | |
| Assay of | 100 | 100 | 99 | 100 | 100 | 100 | 101 | 101 | 101 |
| dibasic | | | | | | | | | |
| potassium | | | | | | | | | |
| phosphate | | | | | | | | | |
| (% w/w) | | | | | | | | | |
| Assay of | 99 | — | — | 99 | — | — | 99 | — | — |
| sodium | | | | | | | | | |
| chloride | | | | | | | | | |
| (% w/w) | | | | | | | | | |
| Osmolality | 407 | 400 | 403 | 405 | 401 | 402 | 408 | 402 | 404 |
| mOsmol/kg | | | | | | | | | |
| Aluminum | 3 | — | 5 | 10 | — | 1 | 9 | — | 2 |
| Content | | | | | | | | | |
| (mcg/ml) | | | | | | | | | |

Data from Table 5 demonstrates excellent storage stability of the ready-to-infuse Formulation 1 under accelerated storage conditions. The formulation exhibits minimal batch-to-batch variability while maintaining the initial pH of 6.6; an assay of monobasic and dibasic potassium phosphate from 99-102% w/w, an osmolality value of from 250-450 mOsmol/kg (e.g., 400-410 mOsmol/kg); and aluminium content of less than 25 mcg/mL (e.g., from 3-10 mcg/mL) during the 6-month storage under accelerated storage conditions.

Table 6 provides 9-month storage stability of terminally sterilized Formulation 1 under long term storage conditions (25° C.±2° C./40% RH±5% RH).

TABLE 6
Potassium phosphates in 0.9% sodium chloride injection, Phosphorus
15 mmol/250 mL and Potassium 22 mEq/250 mL (Formulation 1)

| | Batch 1 | | | | Batch 2 | | | | Batch 3 | | | |
|---|---|---|---|---|---|---|---|---|---|---|---|---|
| Data | Init | 3 M | 6 M | 9 M | Init | 3 M | 6 M | 9 M | Init | 3 M | 6 M | 9 M |
| pH | 6.6 | 6.6 | 6.6 | 6.6 | 6.6 | 6.6 | 6.6 | 6.6 | 6.6 | 6.6 | 6.7 | 6.6 |
| Assay of | 101 | 101 | 101 | 101 | 101 | 101 | 101 | 101 | 102 | 101 | 102 | 102 |
| monobasic | | | | | | | | | | | | |
| potassium | | | | | | | | | | | | |
| phosphate | | | | | | | | | | | | |
| (% w/w) | | | | | | | | | | | | |
| Assay of | 100 | 100 | 100 | 100 | 100 | 100 | 100 | 100 | 100 | 100 | 100 | 100 |
| dibasic | | | | | | | | | | | | |
| potassium | | | | | | | | | | | | |
| phosphate | | | | | | | | | | | | |
| (% w/w) | | | | | | | | | | | | |
| Assay of | 99 | — | — | — | 99 | — | — | — | 99 | — | — | — |
| sodium | | | | | | | | | | | | |
| chloride | | | | | | | | | | | | |
| (% w/w) | | | | | | | | | | | | |
| Osmolality | 407 | 401 | 402 | 403 | 405 | 399 | 401 | 402 | 408 | 403 | 406 | 403 |
| Aluminium | 3 | — | — | — | 10 | — | — | — | 9 | — | — | — |
| Content | | | | | | | | | | | | |
| (mcg/mL) | | | | | | | | | | | | |

Data from Table 6 demonstrates excellent storage stability, with minimal batch-to-batch variability, of the ready-to-use Formulation 1 under long-term storage conditions. The formulations maintain an initial pH of 6.6 during the 6-month storage under long-term storage conditions; maintain an assay of monobasic and dibasic potassium phosphate from 99-102% w/w; maintain an osmolality value of from 250-450 mOsmol/kg (e.g., 390-410); and exhibit an aluminium content of less than 25 mcg/mL (e.g., from 3-10 mcg/mL).

Table 7 provides 6-month storage stability of Formulation 1 under refrigeration (2° C. to 8° C.) and freezing (−20° C.±5° C.) storage conditions.

TABLE 7
Potassium phosphates in 0.9% sodium chloride injection, Phosphorus
15 mmol/250 mL and Potassium 22 mEq/250 mL (Formulation 1)

| | | Refrigeration | | Freezing | |
|---|---|---|---|---|---|
| | | condition | | condition | |
| | | (2° C. to 8° C.) | | (−20° C. ± 5° C.) | |
| Data | Initial | 3 M | 6 M | 3 M | 6 M |
| pH | 6.654 | 6.624 | 6.624 | 6.625 | 6.627 |
| Assay of monobasic | 101.4 | 101.8 | 101.2 | 102.5 | 101.6 |
| potassium Phosphate | | | | | |
| (% w/w) | | | | | |
| Assay of dibasic | 100.5 | 100.9 | 100.0 | 101.0 | 100.5 |
| potassium Phosphate | | | | | |
| (% w/w) | | | | | |
| Assay of sodium chloride | 100.5 | 99.6 | 100.5 | 101.1 | 101.2 |
| (% w/w) | | | | | |
| Osmolality (mOsmol/kg) | 399 | 402 | 405 | 405 | 406 |
| Aluminium Content | 6.06 | — | — | 11.7 | 6.9 |
| (mcg/mL) | | | | | |

Data from Table 7 demonstrates excellent storage stability of ready-to-infuse formulation 1 under refrigeration and freezing storage conditions. The formulation maintains an initial pH of 6.6 during the 6-month storage under refrigerated and freezing storage conditions; maintains an assay of monobasic and dibasic potassium phosphate from 100-102% w/w; maintains an osmolality value of from 250-450 mOsmol/kg (e.g., 390-410); and exhibits an aluminium content of less than 25 mcg/mL.

1. A ready-to-infuse aqueous potassium phosphates injection composition comprising monobasic potassium phosphate, dibasic potassium phosphate, and a tonicity agent, wherein the composition comprises potassium 22 mEq/250 mL and phosphorus 15 mmol/250 mL.
wherein the composition exhibits an osmolality of from about 250 mOsmol/kg to about 450 mOsmol/kg.

2. The composition of claim 1, wherein the composition comprises 0.088 mEq/mL of potassium and 0.06 mmol/mL of phosphorus.

3. The composition of claim 1, wherein the composition is supplied in a bag with a fill volume of about 250 mL.

4. The composition of claim 1, wherein the tonicity agent is selected from the group consisting of sodium chloride, potassium chloride, dextrose, sugars, sugar alcohols, glucose, xylitol, fructose, glycerol, sorbitol, mannitol, mannose, calcium chloride, magnesium chloride, other inorganic salts, and mixtures thereof.

5. (canceled)

6. The composition of claim 1, wherein the composition exhibits a pH of from about 5.5 to about 7.5.

7. The composition of claim 1, wherein the composition upon storage for at least about 6 months at 25° C.±2° C./40% RH±5% RH maintains an the osmolality of from about 250 mOsmol/kg to about 450 mOsmol/kg.

8. The composition of claim 1, wherein the composition exhibits an osmolarity of about 0.455 mOsmol/mL.

9. The composition of claim 1, wherein the composition is supplied in a bag that is further packed in an aluminum overwrap or overpouch.

10. The composition of claim 1, wherein the composition is terminally sterilized.

11. The composition of claim 1, wherein the composition further comprises less than or equal to about 15 mcg/L of aluminum.

12. The composition of claim 7, wherein the composition upon storage for a storage period of at least about 12 months at 25° C.±2° C./40% RH±5% RH, maintains an osmolality value of from about 400 mOsmol/kg to about 410 mOsmol/Kg during the storage period.

13. A ready-to-infuse aqueous potassium phosphates injection composition comprising monobasic potassium phosphate, dibasic potassium phosphate, and a tonicity agent, wherein the composition comprises potassium 22 mEg/250 mL and phosphorus 15 mmol/250 mL,
wherein the composition, upon storage for a period of at least about 6 months at 40° C.±2° C./NMT 25% RH, maintains an osmolality of from about 250 mOsmol/kg to about 450 mOsmol/Kg.

14. The composition of claim 13, wherein the composition comprises 0.088 mEq/mL of potassium and 0.06 mmol/mL of phosphorus.

15. The composition of claim 13, wherein the composition maintains an osmolality value of from about 400 mOsmol/kg to about 410 mOsmol/kg.

16. The composition of claim 13, wherein the composition exhibits an osmolarity of about 0.455 mOsmol/mL.

17. A ready-to-infuse aqueous potassium phosphates injection composition comprising monobasic potassium phosphate, dibasic potassium phosphate, and a tonicity agent, wherein the composition comprises potassium 22 mEq/250 mL and phosphorus 15 mmol/250 mL,
wherein the composition, upon storage for at least about 6 months at about 5° C.±3° C., maintains an osmolality of from about 250 mOsmol/kg to about 450 mOsmol/Kg.

18. The composition of claim 17, wherein the composition comprises 0.088 mEq/mL of potassium and 0.06 mmol/mL of phosphorus.

19. The composition of claim 17, wherein the composition maintains an osmolality value of from about 400 mOsmol/kg to about 410 mOsmol/kg.

20. The composition of claim 17, wherein the composition exhibits an osmolarity of about 0.455 mOsmol/mL.

21. The composition of claim 17, wherein the composition comprises a dose amount of monobasic potassium phosphate and dibasic potassium phosphate and maintains an assay of each of the monobasic potassium phosphate and the dibasic potassium phosphate of at least about 90% w/w of their corresponding dose amounts, upon storage for a storage period of at least about 6 months at 25° C.±2° C./40% RH±5% RH.
